(19) United States
(12) Patent Application Publication
(10) Pub. No.: US 2024/0225429 A1
(43) Pub. Date: Jul. 11, 2024

(54) ENDOSCOPE DISTAL END, ENDOSCOPE, AND METHOD OF PRODUCING ENDOSCOPE

(71) Applicant: OLYMPUS MEDICAL SYSTEMS CORP., Tokyo (JP)

(72) Inventor: Yosuke ISOBE, Tokyo (JP)

(73) Assignee: OLYMPUS MEDICAL SYSTEMS CORP., Tokyo (JP)

(21) Appl. No.: 18/402,827

(22) Filed: Jan. 3, 2024

Related U.S. Application Data
(60) Provisional application No. 63/438,297, filed on Jan. 11, 2023.

(51) Int. Cl. A61B 1/018; A61B 1/00

(52) U.S. Cl. CPC A61B 1/018; A61B 1/0011

(57) ABSTRACT

A distal end body of an endoscope includes: an outer portion defining an internal space and including a partition, the partition having a first through hole, the partition partitioning the internal space into a first space and a second space; a first sealing portion including a second through hole, the first sealing portion being fitted in the first through hole in a state of protruding from the first through hole toward the first space; and an inner portion being fitted in the second through hole.

### CROSS-REFERENCE TO RELATED APPLICATION

This application is based on and claims priority under 35 U.S.C. § 119 to U.S. Provisional Application No. 63/438,297, filed Jan. 11, 2023, the entire contents of which are incorporated herein by reference.

### BACKGROUND

The present disclosure relates to an endoscope distal end, an endoscope, and a method of producing an endoscope.

An endoscope, a flexible thin insertion portion of which is inserted into a body of a subject, such as a human, and that observes the inside of the subject has conventionally been known (for example, refer to Japanese Patent No. 7022610).

In the endoscope described in Japanese Patent No. 7022610, a distal-end rigid portion, a rising mount, and a link mechanism described below are arranged at a distal end of the insertion portion.

In the distal-end rigid portion, a treatment-tool insertion tube in which a treatment tool, such as a puncture needle, is inserted, and a concave portion that communicates with the treatment-tool insertion tube, and that is positioned on a distal end side relative to the treatment-tool insertion tube are formed.

The rising mount is rotatably housed in the concave portion of the distal-end rigid portion so as to be in a rising state (rising position) or in an inverted state (non-rising position) by rotating, and abuts on a treatment tool inserted in the concave portion through the treatment-tool insertion tube, to adjust a protruding direction of the treatment tool from the concave portion.

The link mechanism is a mechanism that converts a linear movement of a wire that moves back and forth according to an operation of an operating portion of the endoscope into a rotation movement, to rotate the rising mount. This link mechanism includes a rotation axis, an arm portion, a housing, a rotation axis seal, and a housing seal described below.

On a side wall of the concave portion of the distal-end rigid portion, a first through hole that communicates with the concave portion is arranged. That is, the side wall partitions into a concave portion inside and a concave portion outside. In the following, the concave portion inside partitioned by the side wall is denoted as first space, and the concave portion outside partitioned by the side wall is denoted as second space.

The rotation axis is inserted into the first through hole, and is connected to the rising mount in the first space.

The arm portion is connected to the rotation axis in the second space, and rotates integrally with the rotation axis according to an operation of the operating portion of the endoscope.

The housing is partially inserted into the first insertion portion, and rotatably supports the rotation axis in the second space.

The rotation axis seal is a sealing portion to maintain a watertight seal between the rotation axis and the housing.

The housing seal is a sealing portion to maintain a watertight seal between the inner surface of the first through hole and the housing.

### SUMMARY

In some embodiments, a distal end body of an endoscope includes: an outer portion defining an internal space and including a partition, the partition having a first through hole, the partition partitioning the internal space into a first space and a second space; a first sealing portion including a second through hole, the first sealing portion being fitted in the first through hole in a state of protruding from the first through hole toward the first space; and an inner portion being fitted in the second through hole.

In some embodiments, an endoscope includes an insertion portion configured to be inserted into a subject. The insertion portion includes a distal end body disposed at a distal end of the insertion portion; a flexible tube disposed proximally relative to the distal end body. The distal end body includes an outer portion defining an internal space and including a partition having a first through hole, the partition partitioning the internal space into a first space and a second space; a first sealing portion including a second through hole, the first sealing portion being fitted in the first through hole in a state of protruding from the first through hole toward the first space; and an inner portion being fitted in the second through hole.

In some embodiments, a method of producing an endoscope includes: fitting a first sealing portion that has a second through hole in a first through hole formed in a partition, the partition partitioning an internal space into a first space and a second space; fitting an inner portion in the second through hole; and biasing the first sealing portion toward an inner surface of the first through hole by the inner portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to an embodiment.

FIG. 2 is a diagram illustrating a configuration of an distal end body.

FIG. 3 is a diagram illustrating a configuration of the distal end body.

FIG. 4 is a diagram illustrating a configuration of the distal end body.

FIG. 5 is a diagram illustrating a configuration of a first sealing portion.

FIG. 6 is a diagram explaining a function of a tapered portion.

FIG. 7 is a flowchart illustrating a method of producing an ultrasound endoscope.

FIG. 8 is a diagram explaining a method of producing an ultrasound endoscope.

FIG. 9 is a diagram explaining a first modification of the embodiment.

FIG. 10 is a diagram explaining a second modification of the embodiment.

FIG. 11 is a diagram explaining the second modification of the embodiment.

FIG. 12 is a diagram explaining a third modification of the embodiment.

FIG. 13 is a diagram explaining the third modification of the embodiment.

FIG. 14 is a diagram explaining the fourth modification of the embodiment.

### DETAILED DESCRIPTION

Hereinafter, forms to implement the disclosure (hereinafter, embodiments) will be explained with reference to the drawings. The embodiments explained below are not intended to limit the disclosure. Furthermore, in descriptions of the drawings, identical reference symbols are assigned to identical parts.

### Schematic Configuration of Endoscope System

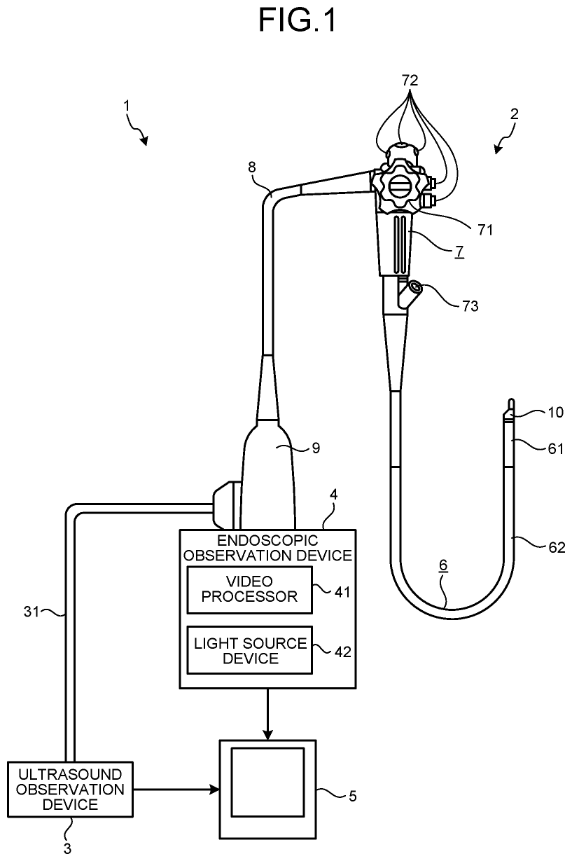

FIG. 1 is a diagram illustrating an endoscope system 1 according to an embodiment.

The endoscope system 1 is a system that performs ultrasound diagnosis of the inside of a subject, such as a human, by using an ultrasound endoscope. This endoscope system 1 includes, as illustrated in FIG. 1, an ultrasound endoscope 2, an ultrasound observation device 3, an endoscopic observation device 4, and a display device 5.

The ultrasound endoscope 2 corresponds to an endoscope. This ultrasound endoscope 2 can be partially inserted into the body of a subject, and has a function of transmitting an ultrasound pulse toward a body wall inside the subject and of receiving an ultrasound echo reflected by the subject to output an echo signal, and a function of outputting an image signal by imaging the inside of the subject.

A detailed configuration of the ultrasound endoscope 2 will be explained in “Configuration of Ultrasound Endoscope” described later.

In the present embodiment, the ultrasound endoscope 2 is adopted as the endoscope, but it is not limited thereto, and an endoscope that does not include an ultrasound probe 11, such as a duodenoscope, may be adopted.

The ultrasound observation device 3 is electrically connected to the ultrasound endoscope 2 through an ultrasound cable 31 (FIG. 1), and outputs a pulse signal to the ultrasound endoscope 2 through the ultrasound cable 31 and receives an echo signal from the ultrasound endoscope 2. In the ultrasound observation device 3, an ultrasonic image is generated by subjecting the echo signal to predetermined processing.

To the endoscopic observation device 4, an endoscope connector 9 (FIG. 1) of the ultrasound endoscope 2 described later is detachably connected. This endoscopic observation device 4 includes, as illustrated in FIG. 1, a video processor 41 and a light source device 42.

The video processor 41 receives an image signal from the ultrasound endoscope 2 through the endoscope connector 9. The video processor 41 generates an endoscopic image by subjecting the image signal to predetermined processing.

The light source device 42 supplies illumination light to illuminate the inside of the subject to the ultrasound endoscope 2 through the endoscope connector 9.

The display device 5 is a display using a liquid crystal or an organic electroluminescence, and displays an ultrasonic image generated by the ultrasound observation device 3 or an endoscopic image generated by the endoscopic observation device 4.

### Configuration of Ultrasound Endoscope

The ultrasound endoscope 2 includes, as illustrated in FIG. 1, an insertion portion 6, an operating portion 7, a universal cord 8, and the endoscope connector 9.

Note that “distal end side” described hereinafter signifies a distal end side of the insertion portion 6 (distal end side in an insertion direction into the subject). Moreover, “proximal end side” described hereinafter signifies a side departing from the distal end of the insertion portion 6.

The insertion portion 6 is a portion that is inserted into the subject. This insertion portion 6 includes, as illustrated in FIG. 1, a distal end body 10, a bendable portion 61, and a flexible tube 62.

In the insertion portion 6, the operating portion 7, the universal cord 8, and the endoscope connector 9, a light guide (not illustrated) that transmits the illumination light supplied from the light source device 42, a transducer cable (not illustrated) that transmits the pulse signal and the echo signal described above, and a signal cable (not illustrated) to transmit an image signal are drawn through, and a lumen (not illustrated) to flow through a fluid is arranged.

The distal end body 10 is arranged at a distal end of the insertion portion 6.

A detailed configuration of the distal end body 10 will be explained in “Configuration of Distal End Body” described later.

The bendable portion 61 is connected to the proximal end side of the distal end body 10, and is bendable.

The flexible tube 62 is connected to a proximal end side of the bendable portion 61, and has flexibility.

The operating portion 7 is connected to the proximal end side of the insertion portion 6, and is a portion that accepts various kinds of operations from a doctor or the like. This operating portion 7 includes, as illustrated in FIG. 1, a bending knob 71 to bend the bendable portion 61, and plural operating portions 72 to perform various operations.

Moreover, in the operating portion 7, a treatment tool inlet 73 that communicates with a tube (not illustrated) arranged inside the bendable portion 61 and the flexible tube 62, and that is to insert a treatment tool (not illustrated) into the tube is arranged.

The universal cord 8 is a cord that extends from the operating portion 7, and in which the light guide (not illustrated) described above, the transducer cable (not illustrated) described above, the signal cable (not illustrated) described above, and the lumen (not illustrated) described above are arranged therein.

The endoscope connector 9 is arranged at an end portion of the universal cord 8. To the endoscope connector 9, the ultrasound cable 31 is connected, and is connected to the video processor 41 and the light source device 42 as it is inserted into the endoscopic observation device 4.

### Configuration of Distal End Body

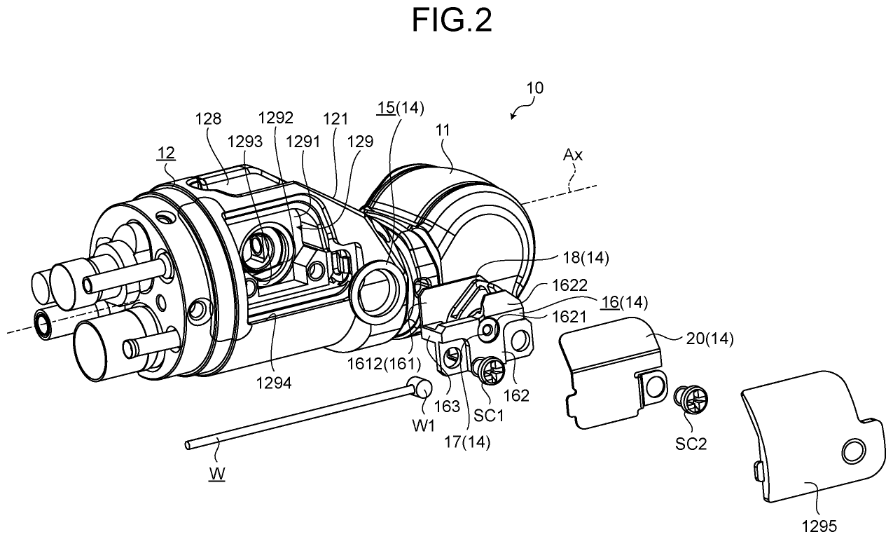

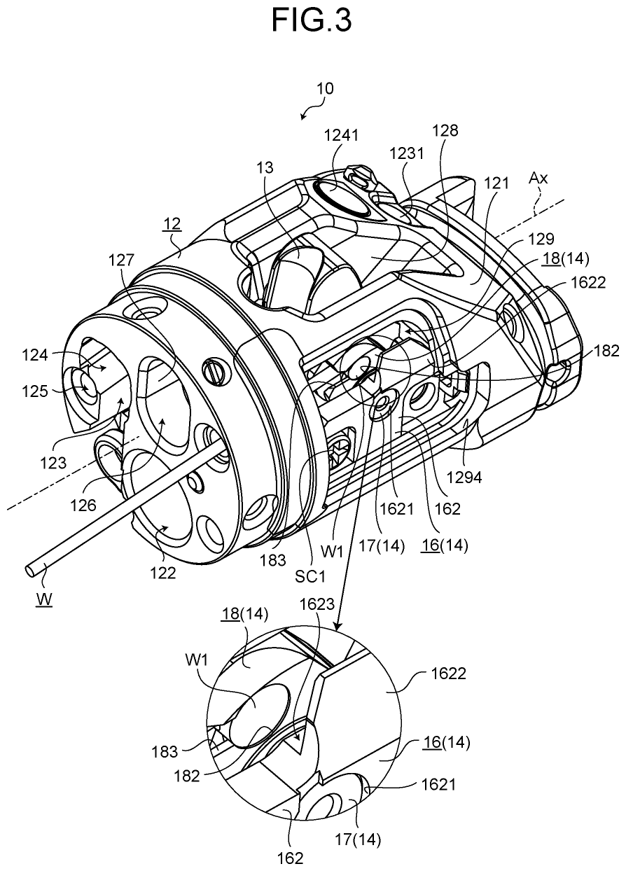

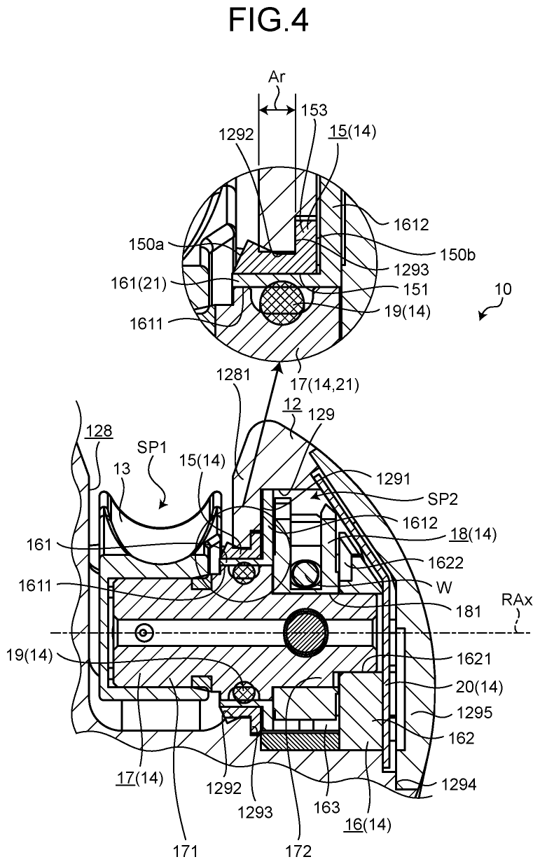

FIG. 2 to FIG. 4 are diagrams illustrating a configuration of the distal end body 10. Specifically, FIG. 2 is an exploded perspective view in which a part of the distal end body 10 is exploded. FIG. 3 is a perspective view of the distal end body 10. FIG. 3 illustrates a state in which the ultrasound probe 11, a cover portion 20, and a cover 1295 are removed. FIG. 4 is a diagram illustrating a part of a cross-section of the distal end body 10 cut along a plane perpendicular to a center axis Ax (FIG. 2, FIG. 3) of the insertion portion 6. The center axis Ax is a center axis of the insertion portion 6 along an extending direction of the insertion portion 6.

The distal end body 10 includes, as illustrated in FIG. 2 to FIG. 4, the ultrasound probe 11 (FIG. 2), a distal-end rigid portion 12, a rising mount 13 (FIG. 3, FIG. 4), and a link mechanism 14.

The ultrasound probe 11 is a convex ultrasound probe, and has plural ultrasound transducers (not illustrated) that are aligned regularly in a state forming a convex arc shape. As the ultrasound probe 11, not limited to the convex ultrasound probe, but also a radial ultrasound probe may be adopted.

The ultrasound transducer has an acoustic lens, a piezoelectric device, and a matching layer, and acquires an ultrasound echo contributing to an ultrasound tomographic image inside the body wall of the subject.

The ultrasound probe 11 converts a pulse signal received from the ultrasound observation device 3 through the ultrasound cable 31 and the transducer cable (not illustrated) described above into an ultrasound pulse, to transmit to the inside of the subject. Moreover, the ultrasound probe 11 converts an ultrasound echo reflected inside the subject into an electrical echo signal, to output to the ultrasound observation device 3 through the transducer cable (not illustrated) described above, and the ultrasound cable 31.

The distal-end rigid portion 12 corresponds to an outer portion. This distal-end rigid portion 12 is a rigid portion constituted of resin material or the like, and has a substantially cylindrical shape extending along the center axis Ax.

In this distal-end rigid portion 12, a slanted surface 121 (FIG. 2, FIG. 3 that shapes the distal-end rigid portion 12 into a tapered shape as it approaches the distal end is arranged on an outer peripheral surface on the distal end side.

In the distal end rigid portion 12, as illustrated in FIG. 3, an attachment hole 122 piercing through from a proximal end to a distal end, an illumination hole 123 piercing through from the proximal end to the slanted surface 121, an imaging hole 124, an air/water supply hole 125, a treatment tool channel 126, and the like are arranged.

The attachment hole 122 is a hole in which the ultrasound probe 11 is attached. Inside the attachment hole 122, the above described transducer cable (not illustrated) that is electrically connected to the ultrasound probe 11 is inserted.

Inside the illumination hole 123, an emitting end side of the light guide (not illustrated) described above, and an illumination lens 1231 (FIG. 3) to irradiate the illumination light emitted from the emitting end of the light guide to the inside of the subject are arranged.

Inside the imaging hole 124, an objective optical system 1241 (FIG. 3) that gathers light (subject image) that has irradiated to the inside of the subject and reflected from the inside of the subject, and an imaging device (not illustrated) that captures a subject image gathered by the objective optical system 1241 are arranged. An image signal captured by the imaging device is transmitted to the endoscopic observation device 4 (video processor 41) through the signal cable (not illustrated) described above.

The air/water supply hole 125 constitutes a part of the lumen (not illustrated) described above, and is a hole to supply air or water to the imaging hole 124, to clean an outer surface of the objective optical system 1241.

The treatment tool channel 126 is a path to let a treatment tool (not illustrated) inserted into the tube (not illustrated) described above inside the insertion portion 6 from the treatment tool inlet 73 protrude outside. This treatment tool channel 126 includes, as illustrated in FIG. 3 and FIG. 4, a treatment-tool insertion hole 127 (FIG. 3) and a housing groove 128.

The treatment-tool insertion hole 127 is apportion that extends from the proximal end toward the distal end side of the distal-end rigid portion 12, and in which a treatment tool (not illustrated) is inserted.

The housing groove 128 is a groove that communicates with the treatment-tool insertion hole 127, and that extends along the center axis Ax from the treatment-tool insertion hole 127 toward the distal end side.

The rising mount 13 is rotatably housed inside the housing groove 128 about a rotation axis RAx (FIG. 4) as illustrated in FIG. 3 and FIG. 4. The rising mount 13 abuts on the treatment tool (not illustrated) inserted in the housing groove 128 through the treatment-tool insertion hole 127, and adjusts a protruding direction of the treatment tool from the housing groove 128.

The link mechanism 14 is a mechanism to rotate the rising mount 13 according to an operation with respect to the operating portion 7 by a doctor or the like.

Hereinafter, a configuration of the link mechanism will be explained.

### Configuration of Link Mechanism

The link mechanism 14 is positioned on a side of the housing groove 128 as illustrated in FIG. 2 to FIG. 4, and is arranged in a concave portion 129 that is recessed toward the housing groove 128 from an outer surface of the distal-end rigid portion 12.

A bottom portion 1291 of this concave portion 129 is constituted of one side wall 1281 (FIG. 4) constituting the housing groove 128. The side wall 1281 is a wall portion perpendicular to the rotation axis RAx, and partitions into an inside of the housing groove 128 and an inside of the concave portion 129. That is, the side wall 1281 corresponds to a partition. Hereinafter, the inside of the housing groove 128 is denoted as first space SP1 (FIG. 4), and the inside of the concave portion 129 is denoted as second space SP2 (FIG. 4).

On the rotation axis RAx of the bottom portion 1291, as illustrated in FIG. 4, a through hole 1292 that pierces through the side wall 1281, and that communicates with the first and the second space SP1, SP2 is arranged. This first through hole 1292 has a planar circular shape coaxial with the rotation axis RAx.

Moreover, in a peripheral portion of the first through hole 1292 on a side of the second space SP2, as illustrated in FIG. 2 or FIG. 4, a groove 1293 in a planar circular shape that is recessed toward the first space SP1, and is coaxial with the rotation axis RAx is arranged.

Furthermore, in an opening 1294 of the concave portion 129, as illustrated in FIG. 2 or FIG. 4, the cover 1295 is mounted. This cover 1295 has a planar shape substantially the same as that of the opening 1294, and closes the concave portion 129 as it is attached to the opening 1294.

The link mechanism 14 includes, as illustrated in FIG. 2 to FIG. 4, a first sealing portion 15 (FIG. 2, FIG. 3), a housing 16, an shaft 17, an arm 18, a second sealing portion 19 (FIG. 4), and the cover portion 20 (FIG. 2, FIG. 4).

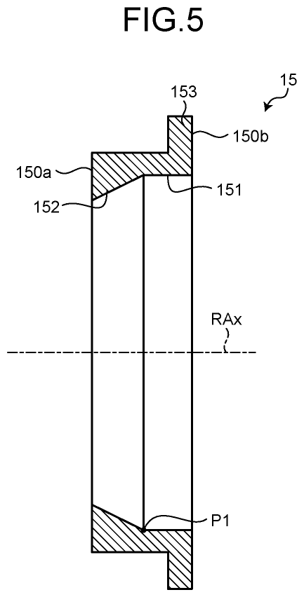

FIG. 5 is a diagram illustrating a configuration of the first sealing portion 15. Specifically, FIG. 5 is a cross-section of the first sealing portion 15 cut along a plane including the rotation axis RAx. FIG. 5 is a diagram illustrating the first sealing portion 15 before performing step S3 described later.

The first sealing portion 15 is constituted of a material having elasticity, such as rubber or silicone, and is a sealing portion that maintains watertight seal between an inner peripheral surface of the first through hole 1292 and the housing 16. This first sealing portion 15 has a annular shape having an outer diameter size substantially the same as an inner diameter size of the first through hole 1292, and is fit in the first through hole 1292 in a state of protruding to the first space SP1 from the first through hole 1292.

Hereinafter, an end portion on the side of the first space SP in the first sealing portion 15 is denoted as first end 150a (FIG. 4, FIG. 5), and an end portion on a side of the second space SP2 is denoted as second end 150b (FIG. 4, FIG. 5).

An inner peripheral surface of the first sealing portion 15 in a annular shape corresponds to a second through hole 151 (FIG. 5).

In the present embodiment, on the inner peripheral surface of the first sealing portion 15, a first narrow portion 152 is arranged as illustrated in FIG. 5.

The first narrow portion 152 corresponds to a narrow portion. This first narrow portion 152 is a portion in which the inner diameter size of the second through hole 151 gradually decreases as it approaches the first space SP1. A position P1 at which the inner diameter size of the second through hole 151 starts decreasing is a substantially center position in a direction along the center axis (rotation axis RAx) in the first sealing portion 15 in FIG. 5, but it is not limited thereto, and it may be any position as long as it is within an area Ar1 (FIG. 4) sandwiched between the inner peripheral surface of the first through hole 1292 excluding the groove 1293 and a first bearing portion 161 described later in the housing 16.

Moreover, on an outer peripheral surface of the first sealing portion 15, a jut-out portion 153 in a planar circular shape that juts out in a diameter direction of the first sealing portion 15 is arranged at the second end 150b. In a state in which the first sealing portion 15 is fit in the first through hole 1292, the jut-out portion 153 is fit in the groove 1293.

The housing 16 is constituted of a rigid material, and is arranged in the second space SP2 by a screw SC1 (FIG. 2). The housing 16 rotatably supports the shaft 17 about the rotation axis RAx. This housing 16 includes, as illustrated in FIG. 2 or FIG. 4, a first bearing portion 161, a second bearing portion 162, and a connecting portion 163.

The first bearing portion 161 has a annular shape and is fit in the second through hole 151 in a state protruding from the second through hole 151 toward the first space SP1. That is, the first bearing portion 161 corresponds to a annular portion. Hereinafter, an inner peripheral surface of the first bearing portion 161 in an annular shape is denoted as first bearing hole 1611 for convenience of explanation. The first bearing portion 161 rotatably supports the shaft 17 inserted in the first bearing hole 1611 about the rotation axis RAx.

On an outer peripheral surface of this first bearing portion 161, a first flange 1612 in a flat plate shape facing the bottom portion 1291 is arranged at an end portion on a side of the second space SP2 as illustrated in FIG. 2 or FIG. 4. This first flange 1612 corresponds to a flange.

The second bearing portion 162 is positioned on a side of the second space SP2 relative to the first bearing portion 161, and faces the first bearing portion 161 while maintaining predetermined spacing therefrom. In this second bearing portion 162, a second bearing hole 1621 in which the shaft 17 is inserted is arranged as illustrated in FIG. 2 or FIG. 4. The second bearing portion 162 rotatably supports the shaft 17 inserted in the second bearing hole 1621 about the rotation axis RAx together with the first bearing portion 161.

In this second bearing portion 162, a second flange 1622 in a flat plate shape facing the first flange 1612 is arranged as illustrated in FIG. 2 to FIG. 4.

The connecting portion 163 is a portion that connects the first and the second bearing portions 161 and 162.

The shaft 17 is constituted of a rigid material, and has a substantially cylindrical shape extending along the rotation axis RAx. This shaft 17 is axially supported in a state of being rotatably inserted in the first and the second bearing holes 1611, 1621 about the rotation axis RAx as illustrated in FIG. 4. Moreover, the shaft 17 has a first protruding portion 171 (FIG. 4) that is arranged protruding to the first space SP1. The first protruding portion 171 is connected to the rising mount 13 arranged in the first space SP1. That is, the shaft 17 rotates integrally with the rising mount 13 about the rotation axis RAX.

The first bearing portion 161 and the shaft 17 explained above correspond to an inner portion 21 (FIG. 4).

The arm 18 is a pillar-shaped object that is constituted of a rigid material, and that extends along the rotation axis RAx. The arm 18 is arranged between the first and the second bearing portions 161 and 162 as illustrated in FIG. 2 or FIG. 4.

In this arm 18, a circular hole 181 (FIG. 4) coaxial with the rotation axis RAx is arranged. The shaft 17 has a second protruding portion 172 (FIG. 4) that is arranged protruding to the second space SP2. The second protruding portion 172 is fixed to the arm 18 in a state of being inserted into the circular hole 181.

Furthermore, in the arm 18, a wire engaging portion 182 that engages with a wire end portion W1 (FIG. 2, FIG. 3) arranged at one end of a wire W that moves back and forth according to an operation with respect to the operating portion 7 by a doctor or the like as illustrated in FIG. 3.

The wire end portion W1 has a cylindrical shape that extends along a direction parallel to the rotation axis RAx. The wire engaging portion 182 has an internal surface shape substantially identical to that of the wire end portion W1, and is constituted of a concave portion that is recessed along a direction parallel to the rotation axis RAx toward the first space SP1 from the second space SP2.

Moreover, in the arm 18, a slit 183 that continues to the wire engaging portion 182 from a side surface of the arm 18, which is a pillar-shaped object, and that is to insert the wire end portion W1 from the side surface to the wire engaging portion 182 is formed as illustrated in FIG. 3.

When the wire W moves back and forth in accordance with a user operation with respect to the operating portion 7 by a doctor or the like, the arm 18 is rotated integrally with the shaft 17 and the rising mount 13 about the rotation axis RAx. That is, as the rising mount 13 is rotated, a protruding direction of a treatment tool (not illustrated) inserted in the housing groove 128 through the treatment-tool insertion hole 127 from the housing groove 128 is adjusted.

To the second flange 1622, a tapered portion 1623 (FIG. 3) is arranged to solve following problems.

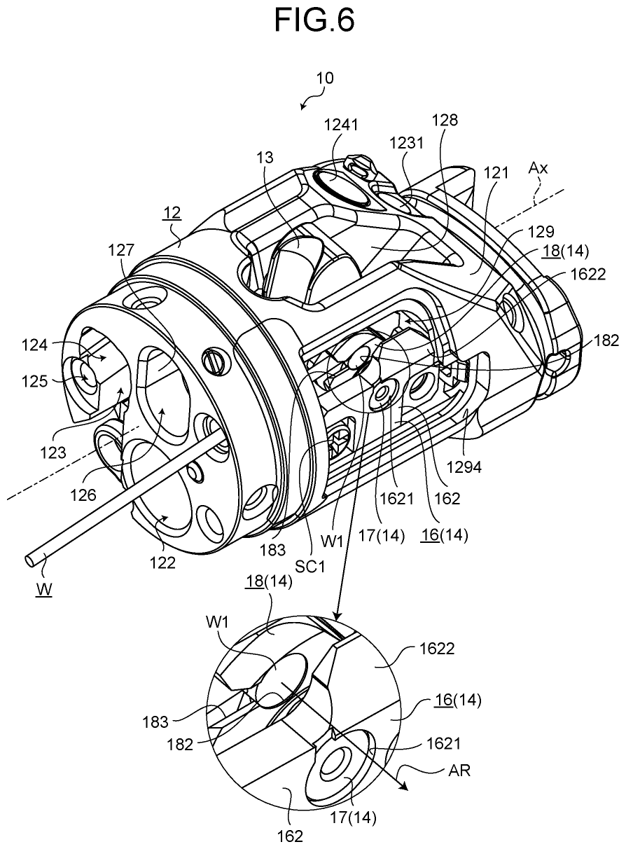

FIG. 6 is a diagram explaining a function of the tapered portion 1623. Specifically, FIG. 6 is a diagram corresponding to FIG. 3, and is a diagram exemplifying a state in which the tapered portion 1623 is not arranged.

For example, when the wire W is bent during assembly, the wire end portion W1 moves toward the second flange 1622 (direction indicated by an arrow AR in FIG. 6) from the wire engaging portion 182, and it can be caught by the second flange 1622. In this case, there is a case in which the rising mount 13 cannot be rotated.

The tapered portion 1623 is a processed portion in a tapered shape arranged at a portion facing the wire end portion W1 in the second flange 1622 to prevent the wire end portion W1 described above from being caught by the second flange 1622.

The second sealing portion 19 is constituted of a material having elasticity, such as rubber or silicone, and is a sealing portion having an annular shape in which the shaft 17 is fit as illustrated in FIG. 4, to maintain watertight sealing between the first bearing portion 161 and the shaft 17.

The cover portion 20 is constituted of a rigid material, and is fixed to the housing 16 by a screw SC2 (FIG. 2) so as to close the concave portion 129.

### Method of Producing Ultrasound Endoscope

Next, a method of producing the ultrasound endoscope 2 will be explained.

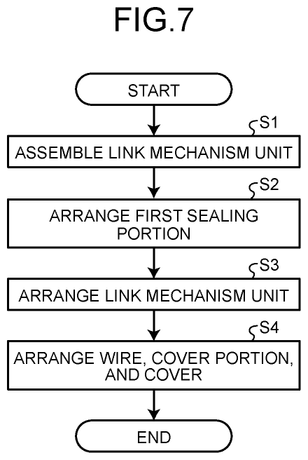

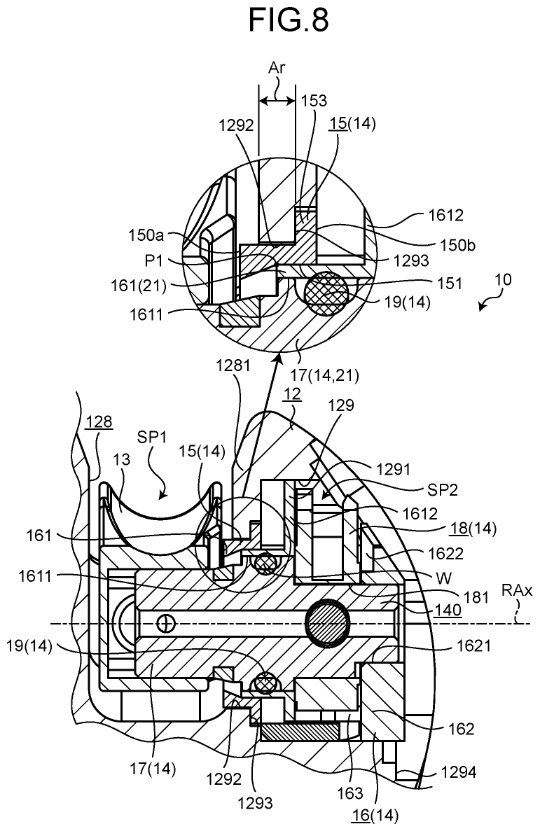

FIG. 7 is a flowchart illustrating a method of producing the ultrasound endoscope 2. FIG. 8 is a diagram explaining the method of producing the ultrasound endoscope 2. Specifically, FIG. 8 is a diagram corresponding to FIG. 4.

In the following, as the method of producing the ultrasound endoscope 2, an assembly method of the distal end body 10 will be explained referring to FIG. 4, FIG. 7, and FIG. 8 for convenience of explanation. FIG. 4 illustrates a state after step S3 is performed. FIG. 8 illustrates a state before step S3 is performed.

First, a worker assembles the housing 16, the shaft 17, the arm 18, and the second sealing portion 19 out of the link mechanism 14 (step S1). Hereinafter, a unit in which the housing 16, the shaft 17, the arm 18, and the second sealing portion 19 are assembled is denoted as link mechanism unit 140 (FIG. 8).

After step S1, the worker fits the first sealing portion 15 in the first through hole 1292 in a state of protruding from the first through hole 1292 toward the first space SP1 as illustrated in FIG. 8 (step S2). At this time, the jut-out portion 153 is fit in the groove 1293.

After step S2, the worker inserts the shaft 17 of the link mechanism unit 140 in the second through hole 151, and fits the first bearing portion 161 in the second through hole 151 in a state of protruding from the second through hole 151 toward the first space SP1 (step S3). At this time, the first bearing portion 161 pushes the first narrow portion 152, and applies, to the first sealing portion 15, a force toward the inner surface of the first through hole 1292. In this state, as illustrated in FIG. 4, the outer diameter size of the first end 150a is to be larger than the inner diameter size of the first through hole 1292. Moreover, the first flange 1612 faces the bottom portion 1291 across the second end 150b. After step S3, the worker connects the shaft 17 with the rising mount 13 in the first space SP1, and arranges the wire end portion W1 in the arm 18, and closes the concave portion 129 with the cover portion 20 and the cover 1295 (step S4).

According to the present embodiment explained above, following effects are produced.

In the method of producing the ultrasound endoscope 2 according to the present embodiment, the first bearing portion 161 of the link mechanism unit 140 is fit in the second through hole 151 (step S3) in a state in which the first sealing portion 15 is fit in the first through hole 1292 in advance (step S2).

Therefore, when assembling the distal end body 10, a case of lacking watertightness due to misalignment of the first end 150a of the first sealing portion 15 from a desired position toward the second space SP2 does not occur.

Therefore, according to the method of producing the ultrasound endoscope 2 according to the present embodiment, assemblability can be improved.

In the first sealing portion 15, the first narrow portion 152 can be arranged.

Therefore, when fitting the first bearing portion 161 of the link mechanism unit 140 in the second through hole 151, the first bearing portion 161 pushes the first narrow portion 152, and a force toward the inner surface of the first through hole 1292 can be applied to the first sealing portion 15. Therefore, a watertight state between the inner surface of the first through hole 1292 and the first bearing portion 161 can be ensured.

Moreover, in the first sealing portion 15, the jut-out portion 153 that is fit in the groove 1293 is arranged.

Therefore, when the first bearing portion 161 of the link mechanism unit 140 is fit in the second through hole 151, it is possible to prevent the first sealing portion 15 from being shifted toward the first space SP1 due to friction from the first bearing portion 161 with the jut-out portion 153. That is, it is possible to prevent the first sealing portion 15 from being shifted from a desired position. Therefore, a watertight state can be ensured between the inner surface of the first through hole 1292 and the first bearing portion 161.

### OTHER EMBODIMENTS

The embodiment of the disclosure has so far been explained, but the disclosure is not to be limited only to the embodiment described above.

In the embodiment described above, the endoscope system 1 has both of the function of generating an ultrasonic image and the function of generating an endoscopic image, but it is not limited thereto, and it may be configured to have only either one of the functions.

In the embodiment described above, not limited to the medical field, the endoscope system 1 may be an endoscope system that observes the inside of a subject, such as a mechanical structure in the industrial field.

In the embodiment described above, configurations of a first to a fourth modifications described below may be adopted.

### First Modification

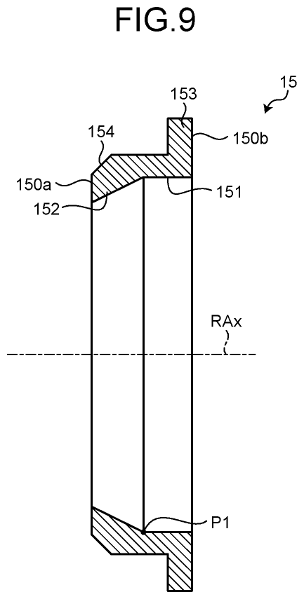

FIG. 9 is a diagram explaining the first modification of the embodiment. Specifically, FIG. 9 is a diagram corresponding to FIG. 5.

In the embodiment described above, a second narrow portion 154 may be arranged on the outer peripheral surface of the first sealing portion 15 as in the first modification illustrated in FIG. 9.

The second narrow portion 154 is a portion in which the outer diameter size of the first sealing portion 15 gradually decreases as it approaches the first space SP1.

According to the first modification explained above, in addition to an effect similar to that of the embodiment described above, a following effect is produced.

In the first sealing portion 15 according to the first modification, the second narrow portion 154 is arranged.

Therefore, a structure easy to fit the first sealing portion 15 in the first through hole 1292 can be implemented.

### Second Modification

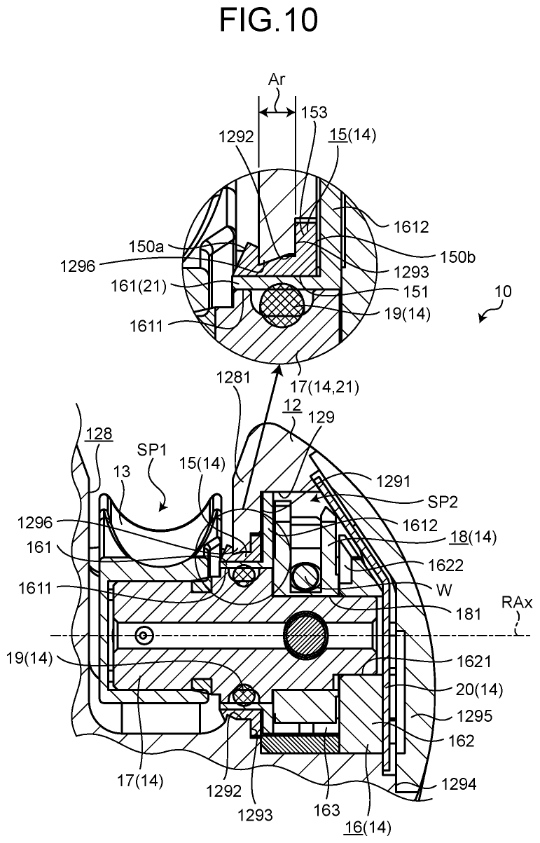

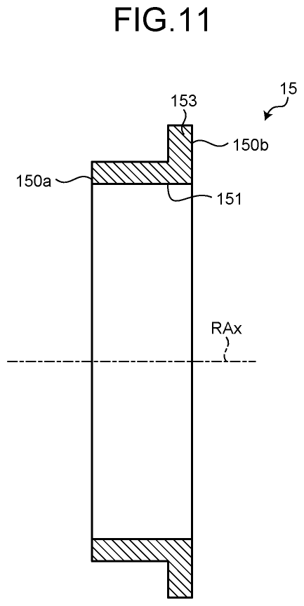

FIG. 10 and FIG. 11 are diagrams explaining the second modification of the embodiment. Specifically, FIG. 10 is a diagram corresponding to FIG. 4. FIG. 11 is a diagram corresponding to FIG. 5.

In the embodiment described above, a configuration of the second modification illustrated in FIG. 10 and FIG. 11 may be adopted.

Specifically, in the first sealing portion 15 according to the second modification, the first narrow portion 152 is not arranged as illustrated in FIG. 11. That is, the inner diameter size of the second through hole 151 is set to be substantially the same at all positions.

Moreover, in the distal-end rigid portion 12 according to the second modification, a first narrow portion 1296 is arranged on the inner peripheral surface of the first through hole 1292 as illustrated in FIG. 10.

The first narrow portion 1296 is a portion in which the inner diameter size of the first through hole 1292 gradually decreases as it approaches the first space SP1 as illustrated in FIG. 10. As the position P1 at which the inner diameter size of the first through hole 1292 starts decreasing, similarly to the embodiment described above, any position is possible as long as it is within the area Ar1 sandwiched between the inner peripheral surface of the first through hole 1292 excluding the groove 1293 and the first bearing portion 161.

Also when the configuration of the second modification explained above is adopted, effects similar to those of the embodiment described above are produced.

### Third Modification

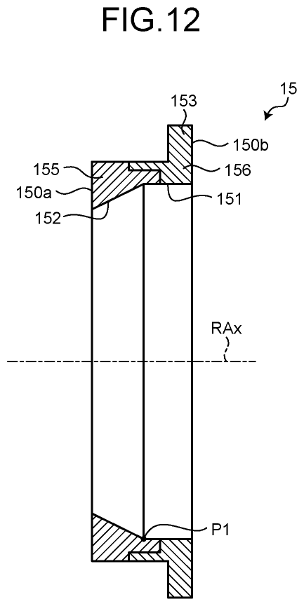

FIG. 12 is a diagram explaining the third modification of the embodiment. Specifically, FIG. 12 is a diagram corresponding to FIG. 5.

In the embodiment described above, as in the third modification illustrated in FIG. 12, the first sealing portion 15 may be constituted of two materials different from each other.

Specifically, the first sealing portion 15 according to the third modification includes, as illustrated in FIG. 12, a first portion 155 that is arranged on the first space SP1 and a second portion 156 that is arranged in the second space SP2.

The second portion 156 is a portion including the second end 150b, and is constituted of a rigid material such as metal.

The first portion 155 is a portion including the first end 150a, is constituted of a material having elasticity, such as rubber or silicone, softer than the second portion 156, and is formed, for example, by insert molding with respect to the second portion 156.

According to the third modification explained above, a following effect is produced in addition to effects similar to those of the embodiment described above.

The first sealing portion 15 according to the third modification includes the second portion 156 constituted of a rigid material such as metal, and the first portion 155 that is formed by, for example, insert molding with respect to the second portion 156.

Therefore, it is possible to improve the strength of the first sealing portion 15. Moreover, it is possible to implement a structure facilitating positioning of the first sealing portion 15 with respect to the first through hole 1292, and positioning of the first bearing portion 161 with respect to the first sealing portion 15.

### Fourth Modification

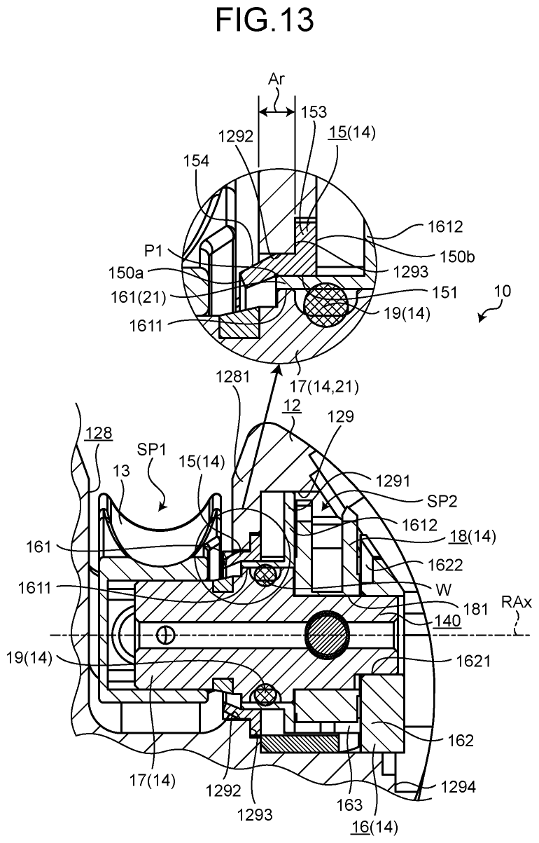

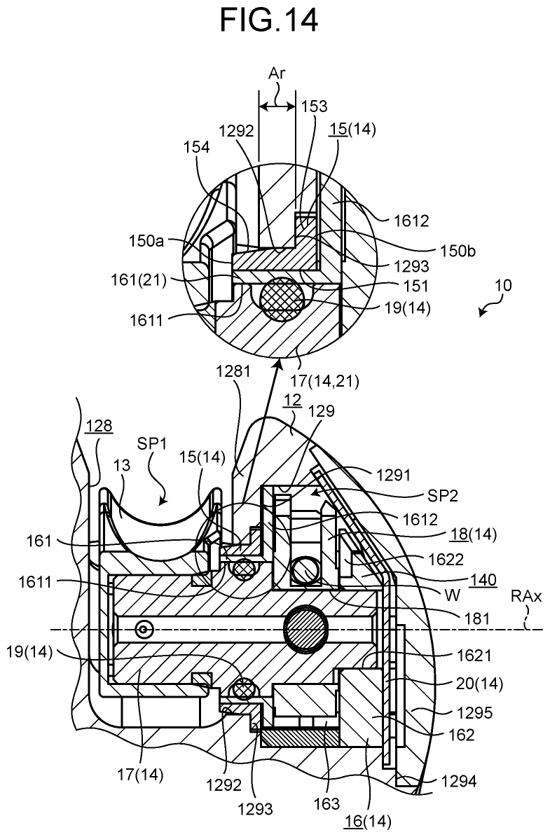

FIG. 13 and FIG. 14 are diagrams explaining the fourth modification of the embodiment. Specifically, FIG. 13 is a diagram corresponding to FIG. 8. FIG. 14 is a diagram corresponding to FIG. 4.

In the embodiment described above, a configuration of the fourth modification illustrated in FIG. 13 and the FIG. 14 may be adopted.

Specifically, the first sealing portion 15 according to the fourth modification has, as illustrated in FIG. 13, the second narrow portion 154 similarly to the first modification described above.

In a state in which the distal end body 10 is assembled, that is, in a state after step S3 is performed, the outer diameter size of the first end 150a is to be the same as or smaller than the inner diameter size of the first through hole 1292 as illustrated in FIG. 14.

Also when the configuration of the fourth modification explained above is adopted, effects similar to those of the embodiment and the first modification described above are produced.

According to an distal end body, an endoscope, and a method of producing an endoscope according to the disclosure, assemblability can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A distal end body of an endoscope comprising:
an outer portion defining an internal space and including a partition, the partition having a first through hole, the partition partitioning the internal space into a first space and a second space;
a first sealing portion including a second through hole, the first sealing portion being fitted in the first through hole in a state of protruding from the first through hole toward the first space; and
an inner portion being fitted in the second through hole.

2. The distal end body according to claim 1, wherein a first end of the first sealing portion on a side of the first space has an outer diameter larger than an inner diameter of the first through hole.

3. The distal end body according to claim 2, wherein the inner portion is fitted in the second through hole in a state of protruding from the second through hole toward the first space.

4. The distal end body according to claim 1, wherein the inner portion includes:
an annular portion fitted in the second through hole in a state of protruding from the second through hole toward the first space; and
a shaft disposed inside the annular portion.

5. The distal end body according to claim 4, further comprising
a second sealing portion sandwiched between the annular portion and the shaft.

6. The distal end body according to claim 4, wherein the annular portion is fitted in the second through hole in a state of protruding from the second through hole toward the second space, and
wherein a flange facing the partition is disposed at an end portion of the annular portion on a side of the second space.

7. The distal end body according to claim 6, wherein the first sealing portion is fitted in the first through hole in a state of protruding from the first through hole toward the second space,
wherein a second end of the first sealing portion on the side of the second space has an outer diameter larger than the inner diameter of the first through hole, and
wherein the flange faces the partition across the second end.

8. The distal end body according to claim 7, wherein the partition has a groove in which the second end is fitted.

9. The distal end body according to claim 4, wherein the shaft is rotatably held with respect to the annular portion, and the shaft is disposed in a state of protruding from an inside of the annular portion toward the first space, and
wherein the shaft includes a first protruding portion protruding toward the first space, and the first protruding portion is connected to a raising base of a treatment tool.

10. The distal end body according to claim 9, wherein the shaft is disposed in a state of protruding from the inside of the annular portion toward the second space, and
wherein the shaft includes a second protruding portion protruding toward the second space, and the second protruding portion is connected to an arm to rotate the shaft.

11. The distal end body according to claim 1, wherein a first end of the first sealing portion on a side of the first space has an outer diameter same as or smaller than an inner diameter of the first through hole.

12. The distal end body according to claim 11, wherein the inner portion is fitted in the second through hole in a state of protruding from the second through hole toward the first space.

13. The distal end body according to claim 1, wherein an outer diameter of a first end of the first sealing portion on a side of the first space is smaller than an outer diameter of a second end of the first sealing portion on a side of the second space.

14. The distal end body according to claim 1, wherein the first sealing portion includes:
a first portion disposed on a side of the first space; and
a second portion formed of a second material different from a first material of the first portion, and the second portion being disposed on a side of the second space.

15. The distal end body according to claim 14, wherein the first material is softer than the second material

16. The distal end body according to claim 15, wherein the first sealing portion is formed by insert molding.

17. An endoscope comprising:
an insertion portion configured to be inserted into a subject, wherein the insertion portion includes:
a distal end body disposed at a distal end of the insertion portion; and
a flexible tube disposed proximally relative to the distal end body, and
the distal end body includes:
an outer portion defining an internal space and including a partition having a first through hole, the partition partitioning the internal space into a first space and a second space;
a first sealing portion including a second through hole, the first sealing portion being fitted in the first through hole in a state of protruding from the first through hole toward the first space; and
an inner portion being fitted in the second through hole.

18. A method of producing an endoscope comprising:
fitting a first sealing portion that has a second through hole in a first through hole formed in a partition, the partition partitioning an internal space into a first space and a second space;
fitting an inner portion in the second through hole; and
biasing the first sealing portion toward an inner surface of the first through hole by the inner portion.

19. The method of producing an endoscope according to claim 18, wherein the first sealing portion has a shape in which a cross-sectional area of a cross-section perpendicular to a center axis of the first through hole increases toward the first space, and
wherein the inner portion fits in the second through hole to bias the first sealing portion toward the inner surface of the first through hole.

20. The method of producing an endoscope according to claim 18, wherein on a side of the first space in the second through hole, a narrow portion having an inner diameter smaller than other portions is disposed, and
wherein the inner portion fits in the second through hole to push the narrow portion and to push the first sealing portion toward the inner surface of the first through hole.
